(19) United States
(12) Patent Application Publication
(10) Pub. No.: US 2024/0091218 A1
(43) Pub. Date: Mar. 21, 2024

(54) METHODS OF TREATING PROSTATE CANCER USING EXICORILANT AND ENZALUTAMIDE

(71) Applicant: Corcept Therapeutics Incorporated, Menlo Park, CA (US)

(72) Inventors: Grace Mann, Menlo Park, CA (US); Joseph Custodio, Menlo Park, CA (US); Iulia Cristina Tudor, Menlo Park, CA (US); Andrew Greenstein, San Francisco, CA (US)

(73) Assignee: Corcept Therapeutics Incorporated, Menlo Park, CA (US)

(21) Appl. No.: 18/239,304

(22) Filed: Aug. 29, 2023

Related U.S. Application Data
(60) Provisional application No. 63/403,478, filed on Sep. 2, 2022.
(60) Provisional application No. 63/414,187, filed on Oct. 7, 2022.
(60) Provisional application No. 63/442,546, filed on Feb. 1, 2023.

(51) Int. Cl. A61K 31/4745; A61K 31/4166; A61P 35/00

(52) U.S. Cl. CPC A61K 31/4745; A61K 31/4166; A61P 35/00

(57) ABSTRACT

Methods of treating prostate cancer, including castration-resistant prostate cancer and metastatic castration-resistant prostate cancer, comprising administering an effective amount of an androgen receptor (AR) antagonist and an effective amount of a nonsteroidal selective glucocorticoid receptor modulator (SGRM) are disclosed. The AR antagonist may be enzalutamide. The SGRM may be an octahydro fused azadecalin compound, such as exicorilant, ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone which has the structure:

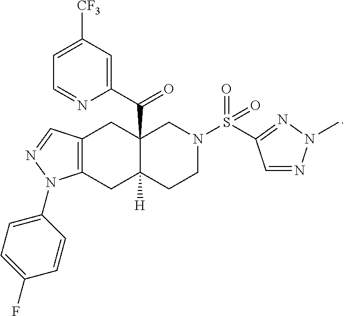 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

The AR antagonist and the SGRM may be administered once per day, and may be administered with food. Enzalutamide doses may be 150-200 mg/day, e.g., 160 mg/day. Exicorilant doses may be 100-350 mg/day, e.g., 240 mg/day, or 280 mg/day, or 320 mg/day.

### CROSS-REFERENCE to RELATED APPLICATIONS

This application claims priority to, and the benefit of, U.S. Provisional Application No. 63/403,478, filed Sep. 2, 2022; U.S. Provisional Application No. 63/414,187, filed Oct. 7, 2022; and U.S. Provisional Application No. 63/442,546, filed Feb. 1, 2023, all of which applications are hereby incorporated by reference herein in their entireties.

### BACKGROUND

Prostate cancer is the most common solid tumor cancer and the second most common cause of cancer-related death in men in the United States. Since prostate cancer growth and proliferation is typically driven by androgens, treatment may include androgen-deprivation, which may include chemical means to reduce androgen levels, to reduce androgen activity (e.g., by inhibiting androgen binding to androgen receptors), and to reduce androgen production in the patient. Such androgen deprivation therapies may be termed “castration” (whether chemical castration or even surgical castration if indicated). However, such treatments are not always successful, or if initially successful, may not remain successful over time. Castration resistant prostate cancer (CRPC) is a serious disease with substantial mortality; in men where the tumor has metastasized (metastatic castration-resistant prostate cancer (mCRPC)) the disease remains incurable and fatal, despite the availability of multiple classes of therapy that delay disease progression and prolong life.

Prostate specific antigen (PSA) is often used as a measure of the activity of prostate tissue; high or rapidly increasing PSA levels in a patient may be a symptom of prostate cancer. Where PSA levels are increasing over time, the time for the PSA level to double (termed “PSA doubling time”) is used as an indicator of prostate tumor growth rate, and may be an indicator of prostate cancer metastasis. Thus, shorter PSA doubling times indicate faster tumor growth than longer PSA doubling times. Increase in the PSA doubling time following treatment indicates that the treatment is having a beneficial effect (e.g., slowing tumor growth or lessening rate of metastasis).

Conventional treatment options for CRPC and mCRPC include androgen-deprivation (“castration”), surgery (at least for primary tumor(s) in the prostate), radiation therapy (also termed “radiotherapy”) and chemotherapy. However, these treatments may not successfully treat the disease. Accordingly, there is need for new and for improved treatments for mCRPC.

### SUMMARY

Disclosed herein are novel methods for treating prostate cancer, including castration resistant prostate cancer (CRPC), comprising administering a selective glucocorticoid receptor modulator (SGRM) to patients receiving androgen receptor (AR) antagonist therapy. In embodiments, the AR antagonist is enzalutamide. In embodiments, the SGRM is exicorilant. Disclosed herein are data from a Phase 1 study of 39 men suffering from castration resistant prostate cancer (CRPC) who received the combination therapy of enzalutamide plus exicorilant. Fourteen patients were enrolled in segment 1 of the study; 25 patients were enrolled in segment 2 of the study.

The SGRM used in the clinical trial disclosed herein is a nonsteroidal compound comprising an octahydro fused azadecalin structure termed “exicorilant” (also known as “CORT125281). Exicorilant is the compound ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone which has the structure:

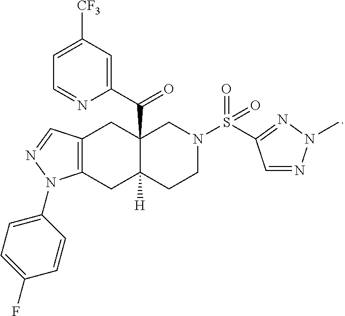 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

The octahydro fused azadecalin structure, and numerous examples of further compounds having an octahydro fused azadecalin structure (which may be termed an “octahydro fused azadecalin backbone”), is described and disclosed in U.S. Pat. No. 10,047,082, the entire contents of which is hereby incorporated by reference in its entirety.

Without being bound by theory, it is hypothesized that blocking an important tumor escape pathway by treating with a SGRM in combination with enzalutamide, an androgen receptor (AR) antagonist, may benefit patients with mCRPC via dual antagonism of the GR and AR receptors. This was the first study evaluating the safety, PK, PD, and preliminary efficacy of exicorilant in combination with enzalutamide in patients with CRPC.

Increases in PSA doubling time were observed in more than 50% of patients dosed daily under fed conditions, indicating that the treatment had a beneficial effect in more than 50% of the treated patients. While baseline 24-hour urinary free cortisol (UFC) values for most patients were within the normal range, improvements in PSA trajectories after treatment with exicorilant in combination with enzalutamide were predominantly observed in the patients with baseline UFC levels greater than 17.5 1.μg/24 hr (P<0.05).

The most frequently reported treatment emergent adverse event (TEAE) assessed as related to exicorilant was fatigue. No grade 4 or 5 exicorilant-related TEAEs were reported in this study. No clinically relevant changes in exposures of enzalutamide or its active metabolite, N-desmethyl enzalutamide, were observed when given in combination with exicorilant, relative to enzalutamide administered alone. Modulation of GR target genes was observed in patients receiving exicorilant. These modulated GR target genes were the same genes that were suppressed in ovarian cancer patients treated with a different SGRM, relacorilant.

### BRIEF DESCRIPTION OF THE DRAWINGS

FIG. 1 shows results of administration of exicorilant (left) and exicorilant plus enzalutamide (right) on prostate cancer growth in mouse xenograft models (mouse 22Rv1 prostate cancer xenografts). Left: N=10 mice/group. Castration performed 5 days after inoculation with 22Rv1 tumor cells; exicorilant treatment initiated on day 6 for 21 days. Right: N=10 mice/group. Castration performed 3 days before implantation of 22Rv1 tumor cells; exicorilant treatment initiated on day 7 for 21 days.

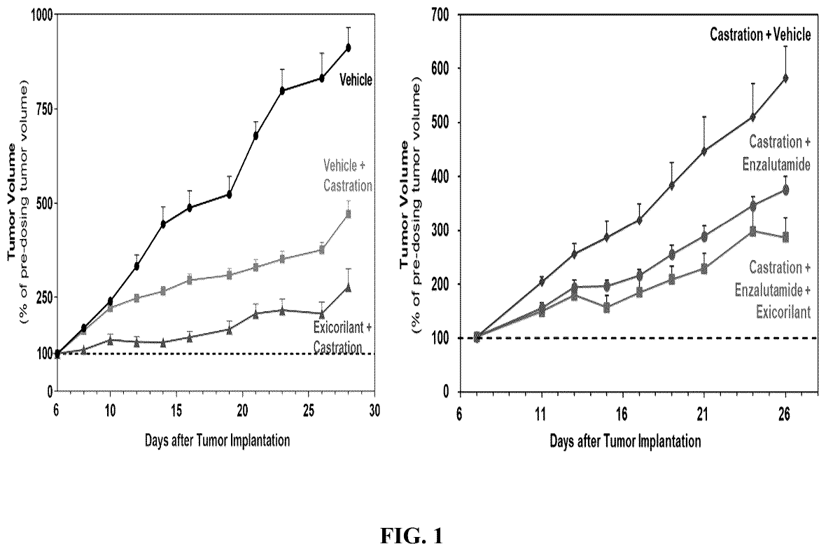

FIG. 2A shows a schematic representation of the clinical study design. In the Figure, “BID” indicates twice per day dose administration; “QD” indicates once per day dose administration; “DRC” Data Review Committee; Note that “CORT125281” is another term for exicorilant. (Study CORT125281-601 is entitled “Study to Evaluate CORT125281 in Combination With Enzalutamide in Patients With mCRPC”, ClinicalTrials.gov Identifier: NCT03437941).

FIG. 2B shows a schematic representation of the Segment 2 clinical study design. Segment 2 of the present study was a double-blind, placebo-controlled phase in patients with rising PSA, in which 25 patients were enrolled and randomized 3:1 to arms A and B.

FIG. 3 provides a table listing demographic information for the patients enrolled in the study.

FIG. 4A illustrates exicorilant pharmacokinetic results in prostate cancer patients receiving enzalutamide and exicorilant.

FIG. 4B illustrates that neither ACTH nor cortisol levels are significantly elevated in prostate cancer patients receiving exicorilant and enzalutamide.

FIG. 5 illustrates CDKN1C expression levels at baseline and after two weeks of 249 milligrams (mg) exicorilant administration in prostate cancer patients receiving enzalutamide and exicorilant.

FIG. 6 illustrates expression levels of selected genes in fasted patients who received exicorilant and enzalutamide BID without food (Segment 1, shown in the left-most graph) and in patients who received exicorilant and enzalutamide twice daily (BID) with food (Segment 2; the following three graphs of the figure).

FIG. 7A illustrates baseline urinary free cortisol (UFC) levels and the prostate specific antigen (PSA) levels in patients receiving exicorilant (EXI) and enzalutamide (ENZA). The left-most columns in the figure answer the yes-or-no question “Is PSA doubling time increased on or after cycle 1 day 1 (C1D1)?”; the middle columns answer the yes-or-no question “Is PSA doubling time increased on or after cycle 2 day 1 (C2D1)?”; and the right-most columns answer the yes-or-no question “Is PSA doubling time increased on or after cycle 3 day 1 (C3D1)?”. Patients with higher baseline UFC levels (greater than 17.5 μg/24 hr) were more likely to experience an increase in PSA doubling time as compared to patients with lower baseline UFC levels.

FIG. 7B shows 24-hour urinary free cortisol (UFC) levels measured in patients after receiving the indicated daily dosages of exicorilant. Exicorilant administration did not increase UFC levels. Data from Segment 2 subjects whose exicorilant doses were escalated to the doses indicated on the horizontal axis. The 24-h UFC reference range was 10-100 μg/day. The 24-h UFC value for C1D1 (cycle 1, day 1) indicates the initial 24-h UFC levels.

FIG. 8A illustrates the best overall response and progression-free survival for patients receiving exicorilant and enzalutamide.

FIG. 8B illustrates reductions in Prostate Specific Antigen (PSA) as compared to baseline PSA measurements.

FIG. 8C illustrates reductions in Prostate Specific Antigen (PSA) at any time during treatment. The lines referred to as “purple” are the darker lines in the figure.

### DETAILED DESCRIPTION

### A. Introduction

The methods disclosed herein can be used to treat a patient suffering from prostate cancer by administering an effective amount of a glucocorticoid receptor modulator (GRM), preferably a selective glucocorticoid receptor modulator (SGRM), in combination with an androgen receptor antagonist effective to treat the prostate cancer. In embodiments, the prostate cancer is castration-resistant prostate cancer. In embodiments, the prostate cancer is metastatic prostate cancer, and may be metastatic castration-resistant prostate cancer (mCRPC). In preferred embodiments, the SGRM is a nonsteroidal SGRM, such as a nonsteroidal SGRM having an octahydro fused azadecalin structure. In embodiments, the nonsteroidal SGRM having an octahydro fused azadecalin structure is exicorilant, which is ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone which has the structure:

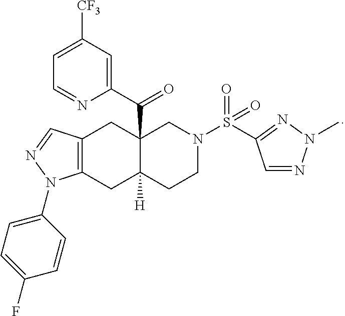 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

Exicorilant is a competitive, reversible, full antagonist of GR (Kᵢ<1 nM in human GR binding and Kᵢ<15 nM in human GR functional assays) with selectivity for GR relative to ER and AR. Suitable doses of exicorilant, in combination with an AR antagonist such as enzalutamide, may be between about 40 milligrams (mg) per day (mg/day) to about 720 mg/day, e.g., between about 200 mg/day (mg/day) and about 350 mg/day; for example, a suitable dose of exicorilant for administration in combination with enzalutamide is 240 mg/day.

In embodiments, the androgen receptor (AR) antagonist is enzalutamide (also known as Xtandi®). Suitable doses of enzalutamide, in combination with exicorilant, may be between about 150 mg/day and about 200 mg/day; for example, a suitable dose of enzalutamide for administration in combination with exicorilant is 160 mg/day.

In mouse xenograft models, exicorilant with castration significantly reduced tumor growth as compared to castration alone. In mouse xenograft models, exicorilant with enzalutamide significantly reduced tumor growth as compared to enzalutamide alone.

Androgen receptor (AR) signaling is a key driver of tumor growth in mCRPC, and AR-targeted therapies are administered to many patients with locally advanced or metastatic disease. Enzalutamide, an androgen receptor (AR) antagonist, is commonly used in such treatments, but resistance to enzalutamide typically develops within 6-12 months. The glucocorticoid receptor (GR) can provide a tumor escape pathway following anti-androgen therapy. For this reason, GR expression in prostate cancer is associated with poor clinical outcomes. Applicants disclose herein the results of a clinical study designed to test the hypothesis that combined administration of a SGRM with an AR antagonist would block this escape pathway via dual antagonism of GR and AR, thereby providing patient benefit. In the present study, mCRPC patients were treated with the SGRM exicorilant in combination with the AR antagonist enzalutamide.

### B. Definitions

As used herein, the term “tumor” and the term “cancer” are used interchangeably and both refer to an abnormal growth of tissue that results from excessive cell division. A tumor that invades the surrounding tissue and/or can metastasize is referred to as “malignant.”

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. For example, a compound or composition may be administered orally to a patient.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art. The effective amount can be an amount effective to invoke an antitumor response. For the purpose of this disclosure, the effective amount of SGRM or the effective amount of an androgen receptor antagonist is an amount that would reduce tumor load or bring about other desired beneficial clinical outcomes related to cancer improvement when combined with an androgen receptor antagonist or SGRM, respectively.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. In some cases, both agents are administered intermittently. In some embodiments, the one pharmaceutical agent, e.g., a SGRM, is administered daily, and the other pharmaceutical agent, e.g., a androgen receptor antagonist, is administered every two, three, or four days.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

A mineralocorticoid receptor (MR), also known as a type I glucocorticoid receptor (GR I), is activated by aldosterone in humans.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. October 1, 2005 35 283-292). The glucocorticoid receptor is also referred to as the cortisol receptor. The term includes isoforms of GR, recombinant GR and mutated GR.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to any composition or compound which modulates GC binding to GR, or modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor modulator binds GR with an affinity that is 100× greater ( 1/100^{th} the Ka value) than its affinity to the MR, AR, or PR, both the MR and PR, both the MR and AR, both the AR and PR, or to the MR, AR, and PR. In another embodiment, the selective glucocorticoid receptor modulator binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRM.

“Glucocorticoid receptor antagonist” (GRA) refers to any compound which inhibits GC binding to GR, or which inhibits any biological response associated with the binding of GR to an agonist. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. A GRA is a compound with an IC50 (half maximal inhibition concentration) of less than 10 micromolar. See Example 1 of U.S. Pat. No. 8,859,774, the entire contents of which is hereby incorporated by reference in its entirety.

As used herein, the term “selective glucocorticoid receptor antagonist” (SGRA) refers to any composition or compound which inhibits GC binding to GR, or which inhibits any biological response associated with the binding of a GR to an agonist (where inhibition is determined with respect to the response in the absence of the compound). By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor antagonist bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor antagonist binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor antagonist binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRA.

Nonsteroidal GRA, SGRA, GRM, and SGRM compounds include compounds comprising a fused azadecalin structure (which may also be termed a fused azadecalin backbone), compounds comprising a heteroaryl-ketone fused azadecalin structure (which may also be termed a heteroaryl-ketone fused azadecalin backbone), compounds comprising an octahydro fused azadecalin structure (which may also be termed an octahydro fused azadecalin backbone), and compounds comprising a pyrimidine cyclohexyl backbone.

Exemplary nonsteroidal GRA, SGRA, GRM, and SGRM compounds comprising a fused azadecalin structure include those described in U.S. Pat. Nos. 7,928,237 and 8,461,172. Exemplary nonsteroidal GRA, SGRA, GRM, and SGRM compounds comprising a heteroaryl-ketone fused azadecalin structure include those described in U.S. Pat. No. 8,859,774. Exemplary nonsteroidal GRA, SGRA, GRM, and SGRM compounds comprising an octahydro fused azadecalin structure include those described in U.S. Pat. No. 10,047,082. Exemplary nonsteroidal GRA, SGRA, GRM, and SGRM compounds comprising a pyrimidine cyclohexyl backbone include compounds disclosed in U.S. Pat. No. 8,685,973. All patents, patent publications, and patent applications disclosed herein are hereby incorporated by reference in their entireties.

Exemplary glucocorticoid receptor antagonists comprising an octohydro fused azadecalin structure include those described in U.S. Pat. No. 10,047,082. In embodiments, the octahydro fused azadecalin is a compound having the formula:

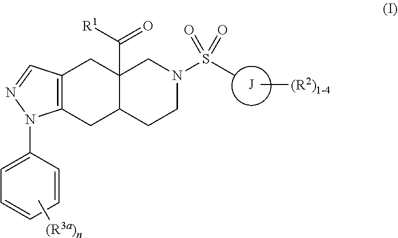

wherein
R¹ is selected from the group consisting of pyridine and thiazole, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of phenyl, pyridine, pyrazole, and triazole;
each R² is independently selected from the group consisting of hydrogen, C₁₋₆ alkyl, halogen, C₁₋₆ haloalkyl, and —CN;
R^{3a} is F;
subscript n is an integer from 0 to 3; or salts and isomers thereof.
   In embodiments, the octahydro fused azadecalin compound is the compound ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone (also termed “exicorilant” or “CORT125281”) which has the structure:

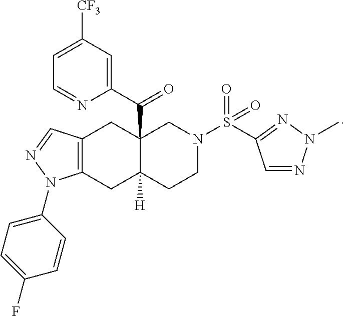 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

In embodiments, the GRM is the nonsteroidal octahydro fused azadecalin GRM compound having the chemical name ((4aR,8aS)-1-(4-fluorophenyl)-6-((2-isopropyl-2H-1,2,3-triazol-4-yl)sulfonyl)-4,4a,5,6,7,8,8a,9-octahydro-1H- pyrazolo[3,4-g]isoquinolin-4a-yl)(thiazol-2-yl)methanone, termed “CORT125329”, having the formula:

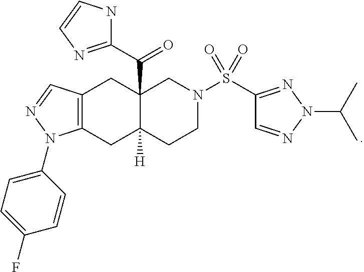 [SMILES: CC(C)n1ncc(S(=O)(=O)N2CC[C@H]3Cc4c(cnn4-c4ccc(F)cc4)C[C@]3(C(=O)c3ncc[nH]3)C2)n1]

As used herein, the term “composition” is intended to encompass a product comprising the specified ingredients such as the said compounds, their tautomeric forms, their derivatives, their analogues, their stereoisomers, their polymorphs, their deuterated species, their pharmaceutically acceptable salts, esters, ethers, metabolites, mixtures of isomers, their pharmaceutically acceptable solvates and pharmaceutically acceptable compositions in specified amounts, as well as any product which results, directly or indirectly, from combination of the specified ingredients in the specified amounts. Such term in relation to a pharmaceutical composition is intended to encompass a product comprising the active ingredient (s), and the inert ingredient (s) that make up the carrier, as well as any product which results, directly or indirectly, in combination, complexation or aggregation of any two or more of the ingredients, or from dissociation of one or more of the ingredients, or from other types of reactions or interactions of one or more of the ingredients. Accordingly, the pharmaceutical compositions of the present invention are meant to encompass any composition made by admixing compounds of the present invention and their pharmaceutically acceptable carriers.

In some embodiments, the term “consisting essentially of” refers to a composition in a formulation whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” can refer to compositions which contain the active ingredient and components which facilitate the release of the active ingredient. For example, the composition can contain one or more components that provide extended release of the active ingredient over time to the subject. In some embodiments, the term “consisting” refers to a composition, which contains the active ingredient and a pharmaceutically acceptable carrier or excipient.

As used herein, “ECOG” refers to the Eastern Cooperative Oncology Group performance score, a measure of patient status (e.g., their ability to care for themselves, daily activity, physical ability, etc.). A score of 0 indicates perfect health, a score of 5 indicates that the patient has died, and intermediate scores indicate levels in between these extremes.

### C. Pharmaceutical Compositions and Administration

In embodiments, the present invention provides a pharmaceutical composition for treating prostate cancer, the pharmaceutical composition including a pharmaceutically acceptable excipient and an octahydro fused azadecalin GRM. In embodiments, the pharmaceutical composition includes a pharmaceutically acceptable excipient and exicorilant. In embodiments, the pharmaceutical composition includes a pharmaceutically acceptable excipient and CORT125329.

In embodiments, the pharmaceutical compositions can be administered orally. For example, the GRM or SGRM can be administered as a pill, a capsule, or liquid formulation as described herein. Alternatively, GRMs can be provided via parenteral administration. For example, the GRM can be administered intravenously (e.g., by injection or infusion). Additional methods of administration of the compounds described herein, and pharmaceutical compositions or formulations thereof, are described herein.

The pharmaceutical compositions can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. In embodiments, the pharmaceutical compositions can also be administered by injection, that is, intravenously, intramuscularly, intracutaneously, subcutaneously, intraduodenally, or intraperitoneally. In embodiments, the pharmaceutical compositions can be administered by inhalation, for example, intranasally. In embodiments, the pharmaceutical compositions can be administered transdermally.

For preparing pharmaceutical compositions from GRMs and SGRMs, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component, a GRM or SGRM. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR modulator mixed with a filler or binders such as lactose or starches, lubricants such as talc or magnesium stearate, and, optionally, stabilizers. In soft capsules, the GR modulator compounds may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use (containing GRMS and SGRMS which are water soluble) can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a SGRM in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

GRMs and SGRMs can be delivered by transdermally, by a topical route, formulated as applicator sticks, solutions, suspensions, emulsions, gels, creams, ointments, pastes, jellies, paints, powders, and aerosols.

GRMs and SGRMs can also be delivered as microspheres for slow release in the body. For example, microspheres can be administered via intradermal injection of drug -containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the invention, for GRMS and SGRMs which form salts, can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component, a GRM or SGRM. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 6000 mg, most typically 50 mg to 500 mg. Suitable dosages also include about 1 mg, 5, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 mg, according to the particular application and the potency of the active component. The composition can, if desired, also contain other compatible therapeutic agents.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In some embodiments, the GRM is administered in one dose. In other embodiments, the GRM is administered in more than one dose, e.g., 2 doses, 3 doses, 4 doses, 5 doses, 6 doses, 7 doses, or more. In some cases, the doses are of an equivalent amount. In other cases, the doses are of different amounts. The doses can increase or taper over the duration of administration. The amount will vary according to, for example, the GRM properties and patient characteristics.

Any suitable GRM dose may be used in the methods disclosed herein. The dose of GRM that is administered can be at least about 300 milligrams (mg) per day, or about 600 mg/day, e.g., about 600 mg/day, about 700 mg/day, about 800 mg/day, about 900 mg/day, about 1000 mg/day, about 1100 mg/day, about 1200 mg/day, or more. For example, where the GRA is mifepristone, the GRM dose may be, e.g., 300 mg/day, or 600 mf/day, or 900 mg/day, or 1200 mg/day of mifepristone. In embodiments, the GRM is administered orally. In some embodiments, the GRM is administered in at least one dose. In other words, the GRM can be administered in 1, 2, 3, 4, 5, 6, 7, 8, 9, 10 or more doses. In embodiments, the GRM is administered orally in 1, 2, 3, 4, 5, 6, 7, 8, 9, 10 or more doses.

The subject may be administered at least one dose of GRM in one or more doses over, for example, a 2-48 hour period. In some embodiments, the GRM is administered as a single dose. In other embodiments, the GRM is administered in more than one dose, e.g. 2 doses, 3 doses, 4 doses, 5 doses, or more doses over a 2-48 hour period, e.g., a 2 hour period, a 3 hour period, a 4 hour period, a 5 hour period, a 6 hour period, a 7 hour period, a 8 hour period, a 9 hour period, a 10 hour period, a 11 hour period, a 12 hour period, a 14 hour period, a 16 hour period, a 18 hour period, a 20 hour period, a 22 hour period, a 24 hour period, a 26 hour period, a 28 hour period, a 30 hour period, a 32 hour period, a 34 hour period, a 36 hour period, a 38 hour period, a 40 hour period, a 42 hour period, a 44 hour period, a 46 hour period or a 48 hour period. In some embodiments, the GRM is administered over 2-48 hours, 2-36 hours, 2-24 hours, 2-12 hours, 2-8 hours, 8-12 hours, 8-24 hours, 8-36 hours, 8-48 hours, 9-36 hours, 9-24 hours, 9-20 hours, 9-12 hours, 12-48 hours, 12-36 hours, 12-24 hours, 18-48 hours, 18-36 hours, 18-24 hours, 24-36 hours, 24-48 hours, 36-48 hours, or 42-48 hours.

Single or multiple administrations of formulations can be administered depending on the dosage and frequency as required and tolerated by the patient. The formulations should provide a sufficient quantity of active agent to effectively treat the disease state. Thus, in one embodiment, the pharmaceutical formulation for oral administration of a GRM is in a daily amount of between about 0.01 to about 150 mg per kilogram of body weight per day (mg/kg/day). In some embodiments, the daily amount is from about 1.0 to 100 mg/kg/day, 5 to 50 mg/kg/day, 10 to 30 mg/kg/day, and 10 to 20 mg/kg/day. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing parenterally administrable formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

The duration of treatment with a GRM or SGRM to treat prostate cancer can vary according to the severity of the condition in a subject and the subject's response to GRMs or SGRMs. In some embodiments, GRMs and SGRMs can be administered for a period of about 1 week to 104 weeks (2 years), more typically about 6 weeks to 80 weeks, most typically about 9 to 60 weeks. Suitable periods of administration also include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 104 weeks. Suitable periods of administration also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, 100, and 104 weeks. Generally administration of a GRM or SGRM should be continued until clinically significant reduction or amelioration is observed. Treatment with the GRM or SGRM in accordance with the invention may last for as long as two years or even longer.

In some embodiments, administration of a GRM or SGRM is not continuous and can be stopped for one or more periods of time, followed by one or more periods of time where administration resumes. Suitable periods where administration stops include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 100 weeks. Suitable periods where administration stops also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 weeks.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR modulator and disease or condition treated.

In some embodiments, co-administration includes administering one active agent, a GRM or SGRM, within 0.5, 1, 2, 4, 6, 8, 10, 12, 16, 20, or 24 hours of a second active agent, such as an androgen receptor antagonist (e.g., enzalutamide). Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a GRM or SGRM has been formulated in an acceptable carrier, it can be placed in an appropriate container and labeled for treatment of an indicated condition. For administration of a GRM or SGRM, such labeling would include, e.g., instructions concerning the amount, frequency and method of administration.

### D. Combination Therapies

Various combinations with a GRM or SGRM and an androgen receptor antagonist may be employed to reduce the tumor load in the prostate cancer patient. By “combination therapy” or “in combination with”, it is not intended to imply that the therapeutic agents must be administered at the same time and/or formulated for delivery together, although these methods of delivery are within the scope described herein. The GRM or SGRM and the androgen receptor antagonist can be administered following the same or different dosing regimen. In some embodiments, the GRM or SGRM and the androgen receptor antagonist is administered sequentially in any order during the entire or portions of the treatment period. In some embodiments, the GRM or SGRM and the anticancer agent is administered simultaneously or approximately simultaneously (e.g., within about 1, 5, 10, 15, 20, 30 minutes or 1 hour of each other). Non-limiting examples of combination therapies are as follows, with administration of the GRM or SGRM and the androgen receptor antagonist for example, GRM or SGRM is “A” and the androgen receptor antagonist is “B”:

A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. Surgical intervention may also be applied in combination with the descirbed therapy.

The present methods can be combined with other means of treatment such as surgery, radiation, targeted therapy, immunotherapy, use of growth factor inhibitors, or anti-angiogenesis factors.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLE

The following example is provided by way of illustration only and not by way of limitation. Those of skill will readily recognize a variety of noncritical parameters which could be changed or modified to yield essentially similar results.

### Example

The present study was an investigation of the safety and efficacy of exicorilant when administered in combination with enzalutamide in the treatment of patients suffering from metastatic castration-resistant prostate cancer (mCRPC). The study was useful in evaluating the dose, and the safety, pharmacokinetics (PK), and pharmacodynamics (PD) of doses of exicorilant when administered in combination with enzalutamide. Patients with histologically confirmed prostate cancer, who had received at least two prior regimens of cytotoxic chemotherapy, were eligible for this study.

Patients with mCRPC or mCRPC with rising prostate-specific antigen (PSA; 25% increase over nadir and absolute value >1 ng/mL) were treated with both exicorilant and enzalutamide (exicorilant+enzalutamide) until disease progression, unmanageable toxicity, or other discontinuation criteria was observed in the patient.

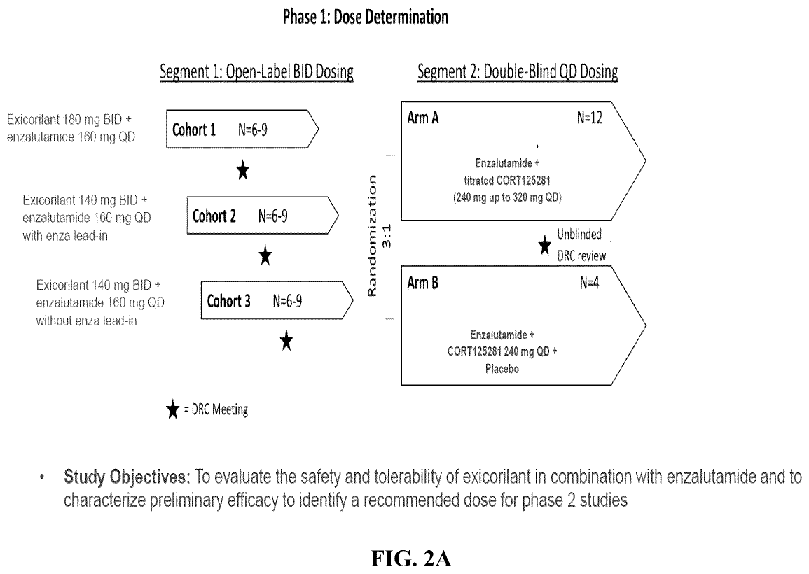

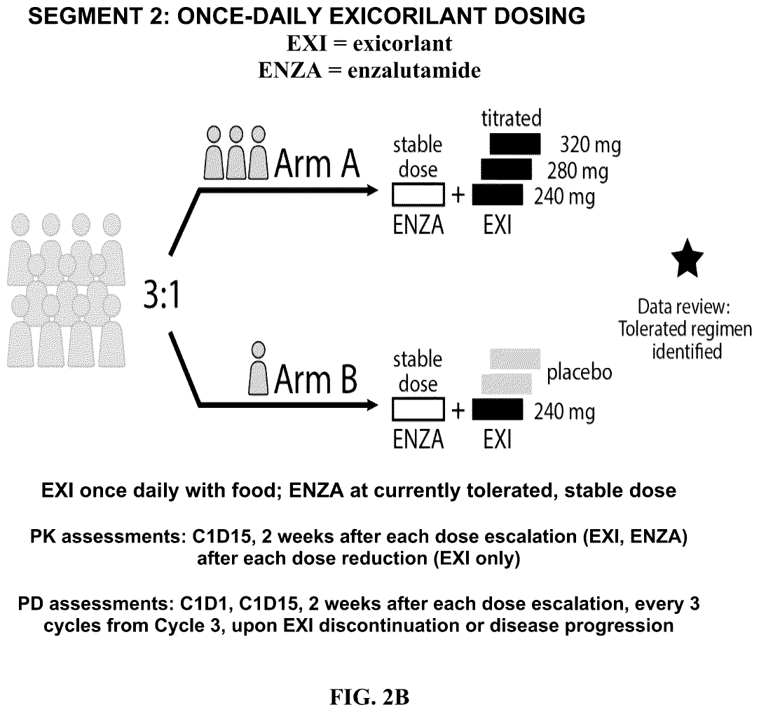

FIGS. 2A and 2B provide schematic representations of the study protocol. As indicated in FIG. 2A, segment 1 of the study was an open-label study in which twice-daily dosing of exicorilant was evaluated. Patients received either exicorilant 180 mg twice daily (BID)+enzalutamide 160 mg once daily (QD), or exicorilant 140 mg BID+enzalutamide 160 mg QD with a 28-day enzalutamide lead-in, or exicorilant 140 mg BID+enzalutamide 160 mg QD without an enzalutamide lead-in. Segment 2 enrolled patients who were on a stable dose of enzalutamide with rising PSA (25% increase over nadir and an absolute value >1 ng/mL) and evaluated QD dosing of exicorilant in a double-blind design. All segment 2 patients received exicorilant 240 mg QD+enzalutamide and were randomized 3:1 to titrate to exicorilant 280 mg or 320 mg or to receive placebo.

As is discussed in more detail in the following, 14 and 25 patients were enrolled in segments 1 and 2, respectively, with 37 patients receiving at least one dose of exicorilant. Most frequent exicorilant-related adverse events (AEs) included fatigue (57%), back pain (35%), decreased appetite (27%), and pain in extremity (22%). Dose-limiting fatigue, musculoskeletal pain, and pancreatitis were observed in segment 1. A phase 2 regimen was selected, for which fatigue and leg/extremity pain consistent with neuropathy were the most common AEs. Other AEs observed in segment 2 were lipase increase, hypophosphatemia, AST/ALT/GGT increase, back pain, and vomiting. Exicorilant exposures were largely overlapping across dose levels in segment 2. Enzalutamide exposures in combination with exicorilant were consistent with historical data for enzalutamide 160 mg alone; no clinically relevant changes in the exposures of enzalutamide or its active metabolite were observed. No clinically relevant changes were observed in enzalutamide exposure when given with exicorilant.

### Exicorilant Dosing Schedules:

Patients in Segment 1 of the study received exicorilant twice daily under fasted conditions. This segment of the study was performed with a standard ‘3+3’ design, including one cohort with an enzalutamide alone lead-in. Enzalutamide was administered once daily.

Patients in Segment 2 of the study received exicorilant once daily under fed conditions. This segment of the study was a double-blind study, in which patients were randomized 3:1 to exicorilant titration (starting at 240 mg, titrated to 280 mg followed by 320 mg every 2 weeks as tolerated starting Cycle 1 Day 16) or to stay on exicorilant 240 mg+placebo. Enzalutamide was administered once daily.

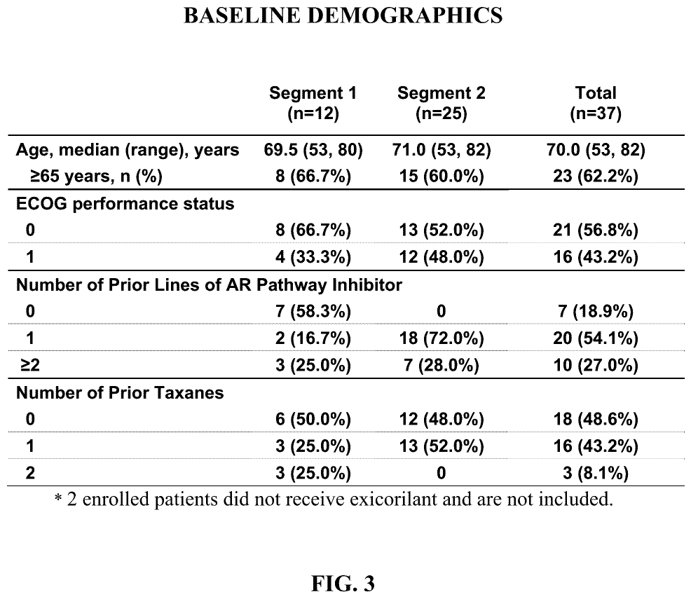

The baseline demographics of patients enrolled in the study are shown in FIG. 3.

Overall safety results for the study are presented in TABLE 1, which shows treatment emergent adverse events (TEAEs) that were reported in more than 10% of patients, and Grade 3 TEAEs reported in more than 1 subject, regardless of causality. TEAEs that were observed in patients treated with exicorilant+enzalutamide overlap with the established adverse event profile of enzalutamide alone. There were no grade 5 adverse events (AEs) and only one grade 4 AE (an unrelated AE of sepsis). (Exicorilant-related TEAEs for Segment 2 alone are reported below in Table 4.)

TABLE 1

| | Segment 1 | | Segment 2 | | Total | |
|---|---|---|---|---|---|---|
| | (N = 12) | | (N = 25) | | (n = 37) | |
| Back pain | 7 | (58.3%) | 17 | (68.0%) | 24 | (64.9%) |
| Fatigue | 6 | (50.0%) | 16 | (64.0%) | 22 | (59.5%) |
| Constipation | 4 | (33.3%) | 10 | (40.0%) | 14 | (37.8%) |
| Decreased appetite | 4 | (33.3%) | 7 | (28.0%) | 11 | (29.7%) |
| Pain in extremity | 3 | (25.0%) | 6 | (24.0%) | 9 | (24.3%) |
| Anemia | 0 | | 8 | (32.0%) | 8 | (21.6%) |
| Abdominal pain | 3 | (25.0%) | 5 | (20.0%) | 8 | (21.6%) |
| Lipase increased | 0 | | 8 | (32.0%) | 8 | (21.6%) |
| Arthralgia | 2 | (16.7%) | 5 | (20.0%) | 7 | (18.9%) |
| Nausea | 2 | (16.7%) | 5 | (20.0%) | 7 | (18.9%) |
| Weight decreased | 3 | (25.0%) | 3 | (12.0%) | 6 | (16.2%) |

Treatment-emergent adverse events (TEAEs) of any grade reported in >15% of patients. There was 1 grade 4 TEAE unrelated to exicorilant (sepsis) and no grade 5 TEAEs.

No clinically relevant changes in the exposures of enzalutamide or its active metabolite, N-desmethyl enzalutamide, were observed in patients receiving exicorilant (the combination of exicorilant+enzalutamide did not change patient exposure to enzalutamide). In Segment 1, enzalutamide-alone exposures (on cycle 1, day 1 (C1D-1) of the lead-in) were compared to exposures following 1 cycle of combination treatment (C2D1). Mean ratios of enzalutamide+N-desmethyl enzalutamide exposures on C2D1 to C1D-1 that were less than 0.75 or that were greater than 1.4 would have been considered indicative of a notable drug-drug interaction. The observed mean ratios of 1.14 (Cₘₐₓ) and 1.26 (AUC₀₋₁₂) do not indicate any notable drug-drug interaction, and indicate that no enzalutamide dose modification would be needed for co-administration of enzalutamide and exicorilant. Thus, these observed mean ratios indicate that a once-daily dose of 160 mg of enzalutamide, when combined with exicorilant would be a safe and effective dose of enzalutamide. Enzalutamide pharmacokinetics in the presence and in the absence of exicorilant are provided in Table 2.

TABLE 2
Enzalutamide pharmacokinetics in the presence
and in the absence of exicorilant

| | C1D-1 | | C2D1 | |
|---|---|---|---|---|
| | (n = 5) | | (n = 4) | |
| | Cₘₐₓ | AUC₀₋₁₂ | Cₘₐₓ | AUC₀₋₁₂ |
| | (ng/mL) | (ng*h/mL) | (ng/mL) | (ng*h/mL) |
| Geometric mean | 15,559 | 145,854 | 18,014 | 187,693 |
| Geometric CV % | 12.9 | 15.7 | 11.3 | 14.0 |

Cₘₐₓ is the maximum observed plasma concentration; AUC₀₋₁₂ is the area under the curve from 0 to 12 hours; C1D-1 is cycle 1 day minus 1 (i.e., the day before cycle 1 day 1); C2D1 is cycle 2 day 1.

Table 2 shows the exposures for enzalutamide+N-desmethyl enzalutamide combined.

Segment 2 of the study was a double-blind, placebo-controlled study of patients with rising PSA; exicorilant was administered once-daily, at a dose that was phased-in (titrated in these patients from 240 mg/day, to 280 mg/ day, to 320 mg/day). The study design of Segment 2 is presented in schematic form in FIG. 2B. Twenty five patients were enrolled and randomized in a ratio of 3:1 to arm A or to arm B. Exicorilant was administered to the patients once per day, with food; enzalutamide was administered at the dose that was currently tolerated and stable for each patient. Pharmacokinetic assessments of both exicorilant and enzalutamide were made on cycle 1, day 15 (C1D15), and two weeks after each dose escalation; pharmacokinetic assessments of exicorilant were made after each dose reduction (if any). Pharmacodynamic (PD) assessments were made on cycle 1, day 1 (C1D1), cycle 1, day 15 (C1D15), and two weeks after each dose escalation every three cycles from cycle 3, and upon exicorilant discontinuation or disease progression.

The baseline demographics of patients enrolled in Segment 2 of the study, and their baseline disease characteristics, are provided in Table 3.

TABLE 3
Baseline Demographics and Disease Characteristics

| | Arm A | | Arm B | | Segment 2 Total | |
|---|---|---|---|---|---|---|
| | (n = 19) | | (n = 6) | | (n = 25) | |
| Disease site, n (%) | | | | | | |
| Lung and/or liver | 4 | (21.1%) | 1 | (16.7%) | 5 | (20.0%) |
| Bone only | 6 | (31.6%) | 2 | (33.3%) | 8 | (32.0%) |
| No measurable disease | 0 | | 1 | (16.7%) | 1 | (4.0%) |
| Median PSA level, | 16.3 | (3.2, 2300) | 14.3 | (6.4, 47.2) | 16.3 | (3.2, 2300) |
| ng/mL median (range) | | | | | | |
| Prior AR-pathway | 19 | (100%) | 6 | (100.0%) | 25 | (100%) |
| inhibitor, n (%) | | | | | | |
| Abiraterone | 4 | (21.1%) | 2 | (33.3%) | 6 | (24.0%) |
| Apalutamide | 1 | (5.3%) | 0 | | 1 | (4.0%) |
| Enzalutamide | 19 | (100%) | 6 | (100.0%) | 25 | (100%) |
| Nilutamide | 3 | (15.8) | 0 | | 3 | (12.0%) |
| Prior taxane, n (%) | 9 | (47.4%) | 4 | (66.7%) | 13 | (52.0%) |
| Cabazitaxel | 1 | (5.3%) | 0 | | 1 | (4.0%) |
| Docetaxel | 8 | (42.1%) | 4 | (66.7%) | 12 | (48.0%) |
| Median Treatment | 6.1 | (2, 61) | 19.5 | (10, 42) | 9.7 | (2, 61) |
| Duration, weeks (range) | | | | | | |

Treatment emergent adverse events (TEAEs) and dose-limiting toxicity events (DLTs) were evaluated for the patients in Segment 2. TEAEs leading to discontinuation of exicorilant included fatigue (n=3), back pain (n=2), pain in extremity (n=2), and groin pain (n=1). Serious adverse events (SAEs) included back pain (n=2), sepsis, confusional state, urinary retention, pelvic pain (n=1 each). Reports of pain in extremity (leg, feet) and sensory neuropathy (legs, feet, toes) were indicative of neuropathic pain. TEAEs of fatigue and back pain, while consistent with enzalutamide treatment and underlying disease, were exacerbated by combination treatment with exicorilant. No SAEs with a fatal outcome were reported. Only 1 SAE was assessed as being related to exicorilant. Three of 25 Segment 2 patient were still receiving exicorilant at the time of tabulation of these results. Most patients discontinued exicorilant due to disease progression or adverse event.

Dose-limiting toxicities (DLTs) were recorded from first dose of exicorilant through Cycle 3 and were defined as considered possibly or probably related to study drug by the investigator. Based on these DLTs, the combined regimen of 240 mg exicorilant once-daily combined with 160 mg enzalutamide once daily was selected as the tolerated phase 2 regimen.

Exicorilant-related TEAEs for the patients in Segment 2 are provided in Table 4.

TABLE 4
Segment 2 Exicorilant-Related TEAEs

All Segment 2 Subjects
(n = 25)

| | Grade ≥3 | All Grades | |
|---|---|---|---|
| Patients with any TEAE | | 23 | (92.0%) |
| related to exicorilant | | | |
| Fatigue | 3 (12.0%) | 16 | (64.0%) |
| Back pain | 2 (8.0%) | 10 | (40.0%) |
| Decreased appetite | — | 6 | (24.0%) |
| Pain in extremity | — | 5 | (20.0%) |
| Constipation | — | 7 | (28.0%) |
| Lipase Increased | 2 (8.0%) | 7 | (28.0%) |
| Nausea | — | 5 | (20.0%) |
| Abdominal pain | 1 (2.7%) | 5 | (20.0%) |
| Anemia | — | 4 | (16.0%) |
| ALT increased | 2 (8.0%) | 3 | (12.0%) |
| ALP increased | — | 3 | (12.0%) |
| Lymphocyte count decreased | — | 3 | (12.0%) |

ALT, alanine aminotransferase; ALP, alkaline phosphatase.
Exicorilant-related TEAs were reported in >15% of patients and grade 3 exicorilant-related TEAs reported in >1 patient. There were no grade 4 or 5 exicorilant-related AEs.

A tabulation of dose-limiting toxicities (DLTs) observed in the patients in Segment 2 is provided in Table 5.

TABLE 5

| | DLT- | | | |
|---|---|---|---|---|
| | Evaluable | Patients | | |
| | Patients | with ≥1 DLT | | DLT |
| Segment 2, n (%) | 16 | 6 | (37.45) | |
| Arm A | 10 | 5 | (50.0%) | Lipase elevation/increase |
| exicorilant 240 mg + | 4 | 4 | (100%) | (n = 3); fatigue, increased ALT |
| enzalutamide 160 mg | | | | (n = 2 each); elevated AST, |
| | | | | increased GGT, worsening |
| | | | | back pain, hypophosphatemia |
| | | | | (n = 1 each) |
| exicorilant 280 mg + | 1 | 1 | (100%) | Back pain, vomiting (n = 1 |
| enzalutamide 160 mg | | | | each) |
| exicorilant 320 mg + | 5 | 0 | | — |
| enzalutamide 160 mg | | | | |
| Arm B | 6 | 1 | (16.7%) | Fatigue |

exicorilant 240 mg +
enzalutamide 160 mg +
Placebo

GGT, gamma-glutamyl transferase.
Exicorilant and Enzalutamide were dosed once daily.

### Pharmacokinetics

Enzalutamide exposures were largely overlapping across Arm A and Arm B, irrespective of exicorilant dose level, and consistent with historical data for enzalutamide 160 mg alone.

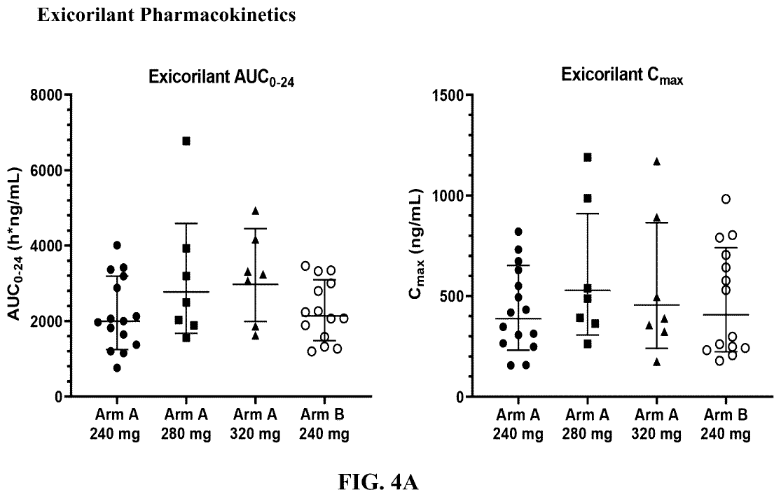

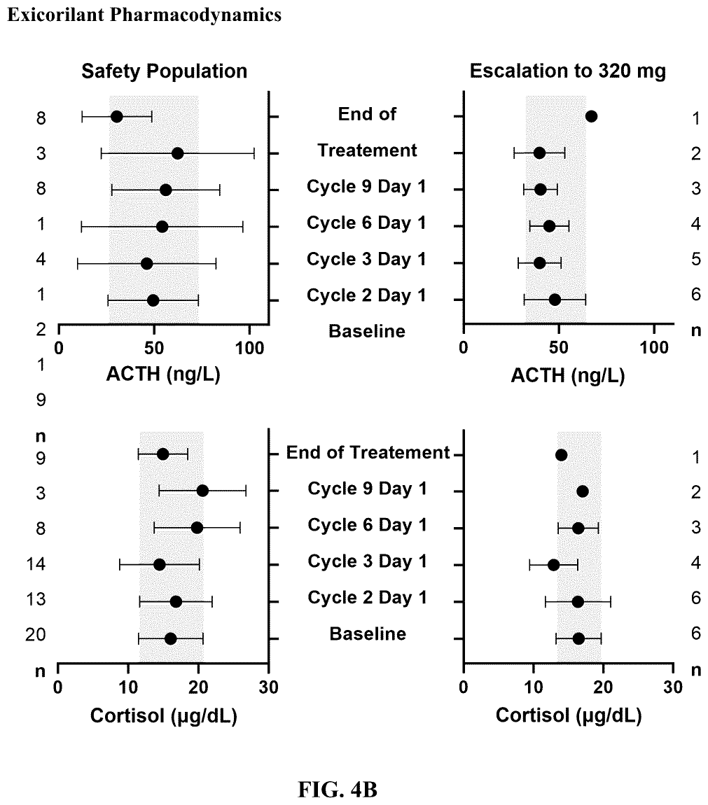

Exicorilant exposures were largely overlapping across arms and dose levels. Greater increases in exicorilant AUC were observed following dose escalation from 240 mg to 280 mg, as compared with 280 mg to 320 mg exicorilant. The mean Cₘₐₓ of exicorilant was similar following 280 mg and 320 mg exicorilant. Exicorilant pharmacokinetics observed in the patients in Segment 2 are illustrated in FIG. 4A (data are shown as geometric mean plus geometric standard deviation), and tabulated in TABLE 6.

TABLE 6

| | Arm A | | | | | | Arm B* | |
|---|---|---|---|---|---|---|---|---|
| | EXI 240 mg + | | EXI 280 mg + | | EXI 320 mg + | | EXI 240 mg + | |
| | ENZA 160 mg | | ENZA 160 mg | | ENZA 160 mg | | ENZA 160 mg | |
| | (15 PK | | (8 PK | | (8 PK | | (14 PK | |
| | observations) | | observations) | | observations) | | observations) | |
| AUC₀₋₂₄ | 366 | (23) | 401 | (15) | 428 | (13) | 375 | (16) |
| (μg · h/mL) | | | | | | | | |
| Cₘₐₓ (μg/mL) | 17.7 | (24) | 19.6 | (20) | 19.6 | (13) | 17.2 | (17) |

Showing geometric mean (geometric CV %).
“EXI” means exicorilant;
“ENZA” means enzalutamide
*Each patient in Arm B had up to 3 pharmacokinetic (PK) observations conducted following exicorilant 240 mg + enzalutamide 160 mg; combined data are presented.

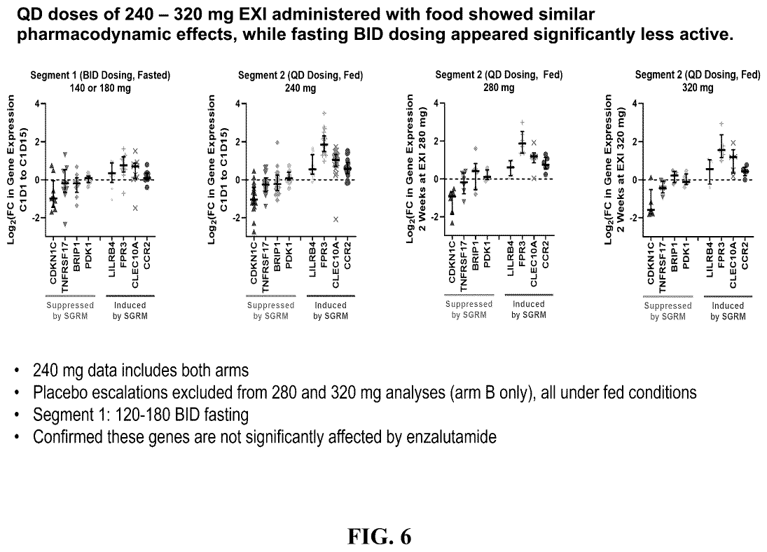

As shown in FIG. 6, exicorilant pharmacodynamic effects (e.g., effects on gene expression) were larger when exicorilant was administered with food, as compared to when exicorilant was administered without food under fasting conditions. Once-daily (QD) doses of 240-320 milligrams (mg) exicorilant administered with food showed similar pharmacodynamic effects, while fasting twice-daily (BID) dosing appeared significantly less active. The 240 mg data in FIG. 6 includes both arms. Placebo escalations were excluded from 280 mg and 320 mg analyses (arm B only), all under fed conditions. Segment 1 was 120-180 mg BID fasting. These results confirm that these genes are not significantly affected by enzalutamide.

### Pharmacodynamics

Consistent with prior studies of exicorilant and other SGRMs, morning serum cortisol and ACTH levels were not affected in the safety population or patients who escalated to 320 mg exicorilant. Thus, exicorilant did not affect cortisol or ACTH levels. See FIG. 4B for graphical representation of these results. FIG. 4B shows the mean±standard deviation (SD) of the adrenocorticotropic hormone (ACTH) and cortisol levels of the patients. The grey band indicates the baseline mean±SD. End of treatment: n=9; cycle 9 day 1: n=3; cycle 6 day 1: n=8; cycle 3 day 1: n=14; cycle 2 day 1: n=13; baseline: n=20.

### Modulation of GR Target Genes Observed

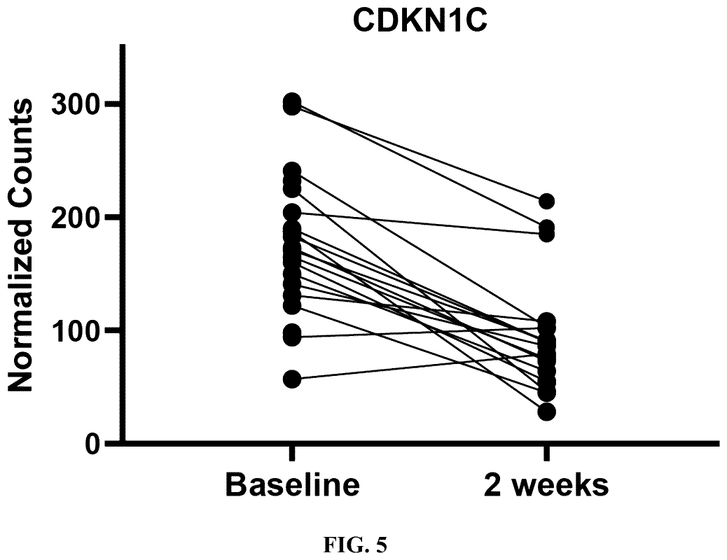

Expression levels of several genes in patients receiving exicorilant were measured by NanoString techniques (NanoString Technologies, Inc., Seattle, WA) in blood. For example, CDKN1C is an established glucocorticoid-inducible gene with important roles in regulating cell growth [Prekovic et al., Nature Communications 4360 (2021)]. Data from the Segment 1 lead-in confirmed that CDKN1C is not affected by enzalutamide alone. Expression levels of CDKN1C were suppressed after 2 weeks of dosing with exicorilant 240 mg+enzalutamide 160 mg (paired T-test P<0.0001). See FIG. 5.

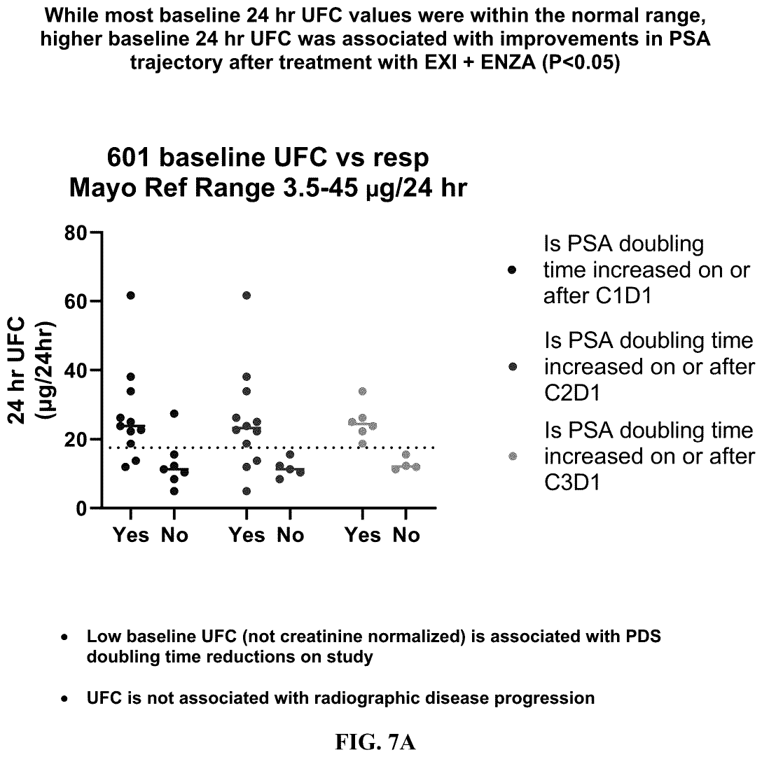

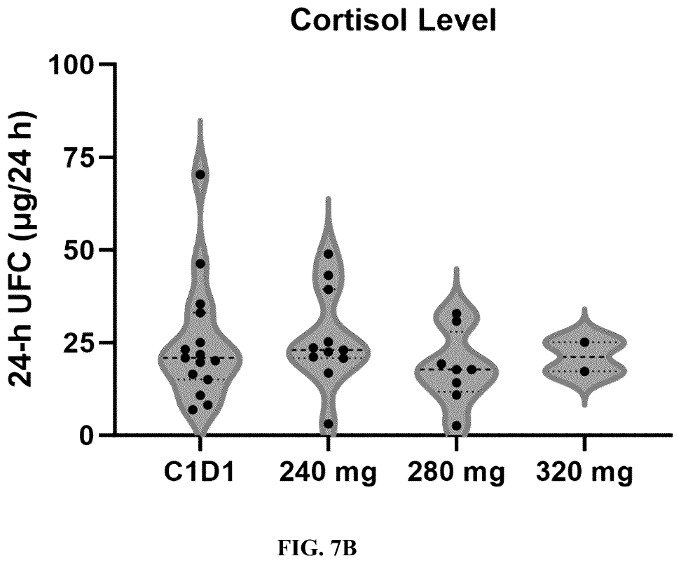

Both segments together enrolled a total of 39 patients (Segment 1: 14, irrespective of prior ENZA exposure; Seg 2: 25, on a stable ENZA dose with rising PSA, defined as a 25% increase over nadir and absolute value >1 ng/mL). Of the 25 patients enrolled in Segment 2, there were no radiographic responses, 18 (72%) patients had a best overall response (BOR) of stable disease per PCWG3 criteria (Prostate Cancer Working Group 3, Scher et al., J Clin Oncol 34:1402-1418 (2016)), and 1 patient achieved a PSA response (≥50% PSA reduction from baseline). Baseline tumor GR expression was detectable in all assessed tumors. High levels of nuclear GR immunoreactivity were observed in nearly all evaluable tumor specimens (n=32), confirming high GR expression in mCRPC patients resistant to AR antagonists (i.e., patients with rising PSA while on enzalutamide). Pharmacodynamic (PD) analyses demonstrated exicorilant modulation of GR target genes, such as CDKN1C. Comparable PD effects were observed across exicorilant doses (240-320 mg QD). While baseline 24-h urinary free cortisol (UFC) for most patients was within the normal range (3.5 to 45 μg/24 h), improvements in PSA trajectories after treatment with exicorilant+enzalutamide were predominantly observed in patients with baseline UFC greater than 17.5 μg/24 hr (P<0.05). (See FIG. 7A.)

As shown in FIG. 7A, while most baseline 24 hour UFC values were within the normal range, higher baseline UFC was associated with improvements in PSA trajectory after treatment with exicorilant plus enzalutamide (P<0.05). Low baseline UFS (not creatinine normalized) is associated with PSA doubling time reductions on study. UFC is not associate with radiographic disease progression. Patients with higher baseline UFC levels (greater than 17.5 μg/24 hr) were more likely to experience an increase in PSA doubling time as compared to patients with lower baseline UFC levels.

As shown in FIG. 7B, exicorilant administration did not increase UFC levels (in those Segment 2 subjects whose exicorilant doses were escalated during the study). Similarly, serum cortisol and ACTH levels were not significantly altered by exicorilant administration. This is in contrast to the effects of the non-selective GR modulator mifepristone, administration of which typically results in up to 3-fold increases in 24-h UFC (Gubbi et al., J Clin Endocrinol Metab 106(5):1501 (2021)).

The time to double PSA levels in a prostate cancer patient provides a measure of tumor progression. A decrease in PSA doubling time (PSADT) indicates more rapid tumor progression; an increase in PSADT indicates a slowing of tumor progression. PSADT increased in 52% of Segment 2 patients (12 of 25) following treatment with exicorilant and enzalutamide. A larger fraction, 61.5%, of Segment 2 patients who received more than two cycles of the combined treatment had a PSADT increase. PSADT was calculated prior to the first dose of exicorilant (patients on enzalutamide alone), and after C1D1, C2D1, and C3D1 (where “C1D1” stands for cycle 1, day 1 of the combined treatment; “C2D1” stands for cycle 2, day 1 of the combined treatment; and “C3D1” stands for cycle 3, day 1 of the combined treatment). Instances of PSADT improvement (increased PSADT) were predominantly observed in patients with higher baseline UFC. Low baseline UFC was associated with PSADT decreases while the patient was on the study treatment.

TABLE 7

| | Pre-Treatment median PSADT, months | 3.6 |
|---|---|---|
| | PSADT increased: | n (%) |
| Segment 2 | After C1D1 (n = 25) | 13 (52.0%) |
| (n = 25) | After C2D1 (n = 23) | 16 (69.6%) |
| | After C3D1 (n = 13) | 8 (61.4%) |

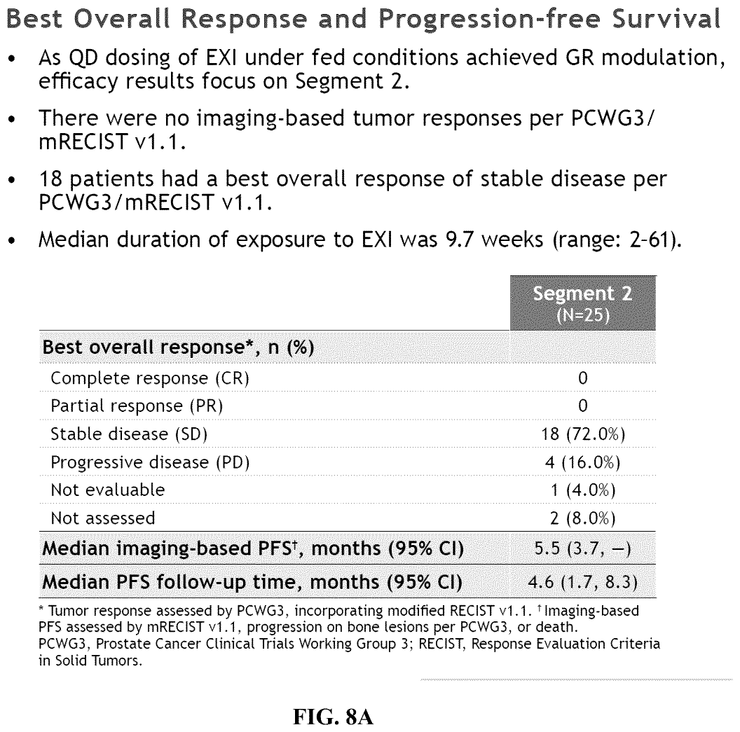

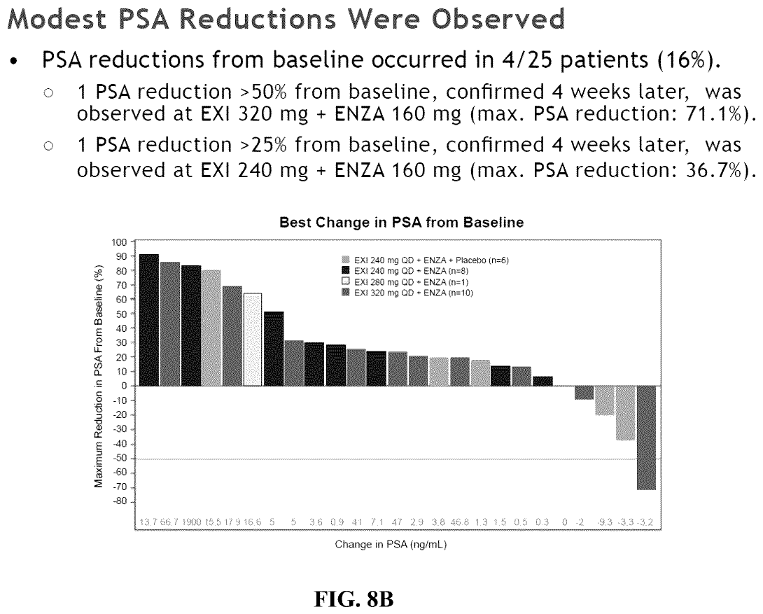

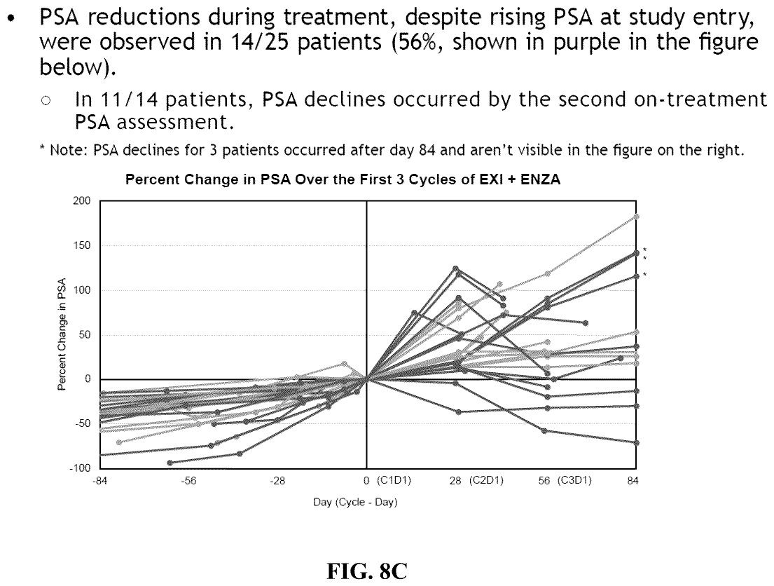

FIG. 8A illustrates the best overall response and progression-free survival for Segement 2 patients receiving exicorilant and enzalutamide. As daily (QD) dosing of exicorilant under fed conditions achieved GR modulation, and as not all patients in Segment 1 were enzalutamide-naïve, efficacy results reported in FIG. 8A focus on Segment 2 patients. The median duration of exposure to exicorilant was 9.7 weeks (range: 2-61). In the results shown in FIG. 8A, tumor response was assessed by Prostate Cancer Clinical Trials Working Group 3 (PCWG3) criteria, incorporating modified “Response Evaluation Criteria in Solid Tumors v1.1” (mRECIST v1.1) criteria. Imaging-based PFS was assessed by mRECIST v1.1, progression on bone lesions per PCWG3, or death. There were no imaging-based tumor responses per PCWG3/mRECIST v1.1 criteria. 18 patients in Segment 2 had a best overall response of stable disease per PCWG3/mRECIST v1.1 criteria.

FIG. 8B illustrates reductions in Prostate Specific Antigen (PSA) as compared to baseline PSA measurements. Reductions in PSA as compared to baseline PSA levels were observed in 15% of the patients (4 of 25). In one patient, a PSA response (defined as a PSA reduction >50% from baseline) was observed in a patient who received 320 mg exicorilant with 160 mg enzalutamide (PSA reduction in this patient: 71.1%). Another patient had a PSA reduction >25% from baseline with 240 mg exicorilant+160 mg enzalutamide (PSA reduction in this patient: 36.7%).

FIG. 8C illustrates reductions in PSA observed at any time during treatment. PSA reductions were observed in 56% (14 of 24 patients) despite rising PSA at entry into the study (indicated by the darker lines in the figure, referred to as “purple” in the bulleted line in the title). In 11 of 14 patients, PSA declines occurred by the second on-treatment PSA assessment. Note: not shown in FIG. 8C are reductions in PSA levels for 3 patients that occurred after day 84, since those data points would be outside the right-most graph limits.

PD biomarker analyses confirmed modulation of GR target genes, such as CDKN1C, by exicorilant. Comparable PD effects were observed across exicorilant doses of 240-320 mg once-daily (QD) with food. These effects were greater than those observed in fasted patients who were administered exicorilant without food (Segment 1 patients).

The results presented in this Example demonstrate that exicorilant in combination with enzalutamide was tolerated and biologically active (e.g., capable of GR modulation) in patients with prostate cancer. The combination consisting of an exicorilant dose of 240 mg/day combined with 160 mg/day enzalutamide was identified by the initial tests as being suitable for administration of combined exicorilant plus enzalutamide for treating patients with prostate cancer. As noted above, the most common TEAEs were fatigue and back pain; however, whether or not such TEAEs might be attributed to exicorilant, or instead might be due to enzalutamide treatment and underlying disease, remains unclear. Applicant again notes that no clinically relevant changes were observed in enzalutamide exposure when given in combination with exicorilant, and that there were no significant alterations in either cortisol levels or ACTH levels due to the administration of exicorilant.

As illustrated in Table 7 and in FIG. 8A, 8B, and 8C, combined treatment with exicorilant and enzalutamide was effective in some patients to slow prostate cancer progression. In the Segment 2 population of heavily pre-treated patients receiving enzalutamide with rising PSA, one PSA response (greater than 50% reduction in PSA), and modest PSA improvements in some other patients, were observed with the addition of exicorilant to the enzalutamide treatment. As shown in Table 7, combined exicorilant and enzalutamide treatment increased PSA doubling times (PSADT) in more than half of the patients in Segment 2, indicating a slowing of disease progression. Two of these patients experienced a greater than 25% increase in PSADT. As shown in FIGS. 8A, 8B, and 8C, more than half the Segment 2 patients had a decrease in PSA at some time during the study. PSA levels in four of 25 (16%) Segment 2 patients were reduced to below baseline PSA levels during the study. One patient experienced a 71.1% reduction in PSA, while one other patient experienced a 36.7% PSA reduction, as compared to their respective baseline PSA levels. These data indicate that combined exicorilant with enzalutamide treatment may be beneficial to patients suffering from histologically confirmed prostate cancer.

All patents, patent publications, publications, and patent applications cited in this specification are hereby incorporated by reference herein in their entireties as if each individual publication or patent application were specifically and individually indicated to be incorporated by reference.

1. A method of treating a patient suffering from prostate cancer, comprising administering to the patient an effective amount of an androgen receptor antagonist and an effective amount of a nonsteroidal selective glucocorticoid receptor modulator (SGRM).

2. The method of claim 1, wherein the patient suffers from castration-resistant prostate cancer.

3. The method of claim 1, wherein the patient suffers from metastatic castration-resistant prostate cancer.

4. The method of claim 1, wherein the androgen receptor antagonist is enzalutamide.

5. The method of claim 1, wherein the SGRM is an octahydro fused azadecalin compound having the formula: 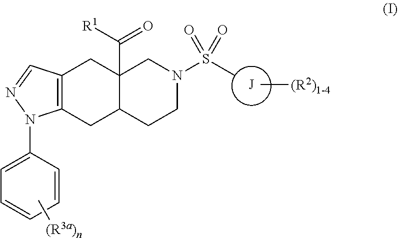
wherein
R¹ is selected from the group consisting of pyridine and thiazole, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of phenyl, pyridine, pyrazole, and triazole;
each R² is independently selected from the group consisting of hydrogen, C₁₋₆ alkyl, halogen, C₁₋₆ haloalkyl, and —CN;
R^{3a} is F;
subscript n is an integer from 0 to 3;
or salts and isomers thereof.

6. The method of claim 5, wherein the octahydro fused azadecalin compound is exicorilant, ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone which has the structure:

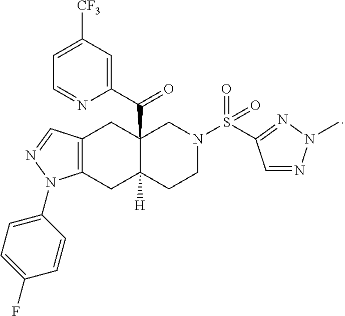 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

7. The method of claim 4, wherein the octahydro fused azadecalin compound is exicorilant, ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone which has the structure:

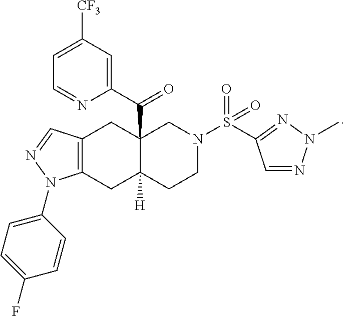 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

8. The method of claim 1, wherein the androgen receptor antagonist and the SGRM are administered to the patient once per day.

9. The method of claim 1, wherein the GRM is administered with food.

10. The method of claim 1, wherein the androgen receptor antagonist is enzalutamide, and the enzalutamide dose is between about 150 milligrams per day (mg/day) and about 200 mg/day.

11. The method of claim 10, wherein the enzalutamide dose is 160 mg/day.

12. The method of claim 1, wherein the SGRM is exicorilant, and the exicorilant dose is between about 100 mg/day and about 350 mg/day.

13. The method of claim 1, wherein the exicorilant dose is selected from the group of exicorilant doses consisting of 140 mg/day, 180 mg/day, 240 mg/day, 280 mg/day, and 320 mg/day.

14. The method of claim 11, wherein the exicorilant dose is selected from the group of exicorilant doses consisting of 140 mg/day, 180 mg/day, 240 mg/day, 280 mg/day, and 320 mg/day.

15. The method of claim 1, wherein the baseline urinary free cortisol (UFC) level of the patient is greater than 17.5 μg/24 hr.

16. The method of claim 9, wherein the baseline urinary free cortisol (UFC) level of the patient is greater than 17.5 μg/24 hr.

17. The method of claim 11, wherein the baseline urinary free cortisol (UFC) level of the patient is greater than 17.5 μg/24 hr.

18. The method of claim 12, wherein the baseline urinary free cortisol (UFC) level of the patient is greater than 17.5 μg/24 hr.

19. The method of claim 2, wherein the androgen receptor antagonist is enzalutamide, and the enzalutamide dose is 160 mg/day; and wherein the SGRM is exicorilant, and the exicorilant dose is between about 100 mg/day and about 350 mg/day.

20. The method of claim 19, wherein the exicorilant dose is selected from the group of exicorilant doses consisting of 140 mg/day, 180 mg/day, 240 mg/day, 280 mg/day, and 320 mg/day.
